(19) United States
(12) Patent Application Publication
(10) Pub. No.: US 2017/0354380 A1
(43) Pub. Date: Dec. 14, 2017

(54) PORTABLE ELECTRONIC DEVICES AND SYSTEMS FOR ANALYZING AN ANALYTE

(71) Applicant: Carydean Enterprises LLC, Milford, NJ (US)

(72) Inventor: Dean Henry Wagstaff, Milford, NJ (US)

(21) Appl. No.: 15/668,318

(22) Filed: Aug. 3, 2017

Related U.S. Application Data
(63) Continuation of application No. 15/618,963, filed on Jun. 9, 2017, now abandoned.
(60) Provisional application No. 62/348,501, filed on Jun. 10, 2016.

(51) Int. Cl. A61B 5/00; A61B 5/157; A61B 50/30; G01N 21/78; G01N 27/416; A61B 5/151; G01N 33/66

(52) U.S. Cl. CPC A61B 5/6898; A61B 5/151; A61B 5/157; G01N 33/66; G01N 21/78; G01N 27/4166; A61B 50/3001; A61B 2562/0295

(57) ABSTRACT

Some embodiments are directed to a portable electronic device for analyzing an analyte. The portable electronic device includes a housing, an adapter detachably coupled to the housing and a processor disposed in the housing. The adapter includes a body defining an opening for receiving a test strip and an interface port disposed within the body. The interface port is configured to read a signal from the test strip. The processor is communicably coupled to the interface port. The processor is configured to determine at least one parameter of the analyte based on the signal received from the interface port.

### CROSS-REFERENCE TO RELATED APPLICATIONS

This application is a continuation of U.S. patent application Ser. No. 15/618,963, filed on Jun. 9, 2017, which claims the benefit of U.S. Provisional Application Ser. No. 62/348,501 filed on Jun. 10, 2016, the entire content of which are hereby incorporated by reference in their entirety.

### FIELD

Embodiments of the present invention generally relate to systems for analyzing an analyte. Specifically, the present invention relates to a portable electronic device for analyzing an analyte.

### BACKGROUND

Due to changing lifestyles, medical issues pertaining to diabetes, hypertension, and high cholesterol are increasing. Various health monitoring devices or analyte sensing devices are typically utilized to monitor parameters related to such medical issues.

Among prevalent medical issues, diabetes has become a major health concern worldwide. Patients are required to regularly monitor and manage their blood glucose levels for managing and controlling the disease. Various glucose meters are well known in the medical industry to measure and monitor one's blood glucose levels. Typically, a pricking needle or a lancet is used to prick the skin of a patient. A droplet of blood is placed onto a sensor strip that is placed in an analyte sensing device. A chemical reaction occurs in the sensor strip and data, i.e., blood glucose level, is generated, which is then displayed on the measuring device indicating the blood glucose level of the user. Moreover, in some glucose measuring devices, the data can also be sent to other devices such as a computer or a cell phone.

However, conventional glucose measuring devices and/or analyte sensing devices are bulky and difficult to carry everywhere. Further, conventional devices for analyte measurement include test insertion ports for only a specific type of sensor strip, making the device incompatible for other types of sensor strips.

Therefore, there is a need to develop an analyte sensing device, such as a portable glucose measuring device, that is compatible with multiple types of sensor strips.

### SUMMARY

Embodiments in accordance with the present invention provide a portable electronic device for analyzing an analyte. The portable electronic device includes a sensor for reading a signal from a test strip including drops of a sample and a processor for determining a parameter of the analyte based on the read signal.

Embodiments in accordance with the present invention provide a portable electronic device for analyzing an analyte, such as measuring glucose levels of blood. The portable electronic device may include adaptor ports of various sizes or a universal adapter port to accommodate multiple test strips from different manufacturers.

Embodiments in accordance with the present invention provide a portable electronic device that transmits data, obtained by measuring blood glucose level, to other devices including, but not restricted to, a computer, tablet or a cell phone via short range wireless communication, such as Bluetooth™.

Embodiments in accordance with the present invention provide a portable electronic device that provides notifications and alerts related to, but not restricted to, high and low blood glucose levels, A1C, parental, endocrinologist and diabetic educators.

Embodiments in accordance with the present invention provide a portable electronic device having one or more compartments for storing multiple test strips, multiple lancets and multiple lancet needles.

In another embodiment of the present invention, the portable electronic device comprises a compartment to accept a separate lancet device containing multiple test strips, multiple lancets and multiple lancet needles.

Embodiments in accordance with the present invention provide a portable electronic device that is wearable. The wearable device can be detachably associated with a band or a strap to be tied on a user's body.

Some embodiments are directed to a portable electronic device for analyzing an analyte. The portable electronic device includes a housing, an adapter detachably coupled to the housing and a processor disposed in the housing. The adapter includes a body defining an opening for receiving a test strip and an interface port disposed within the body. The interface port is configured to read a signal from the test strip. The processor is communicably coupled to the interface port. The processor is configured to determine at least one parameter of the analyte based on the signal received from the interface port.

Some other embodiments are directed to a system for analyzing an analyte. The system includes a portable electronic device and a mobile device communicably coupled to the portable electronic device. The portable electronic device includes a housing, an adapter detachably coupled to the housing and a processor disposed in the housing. The adapter includes a body defining an opening for receiving a test strip and an interface port disposed within the body. The interface port is configured to read a signal from the test strip. The processor is communicably coupled to the interface port. The processor is configured to determine at least one parameter of the analyte based on the signal received from the interface port. The mobile device displays indicia indicative of the at least one parameter of the analyte on a user interface.

Yet other embodiments are directed to a system for analyzing an analyte. The system comprises a portable electronic device and a plurality of adapters. The portable electronic device includes a housing including an adapter port and a processor disposed in the housing and communicably coupled to the adapter port, the processor configured to determine at least one parameter of the analyte based on a signal received from the adapter port. Each of the plurality of adapters is selectively coupled to the adapter port of the housing, each of the plurality of adapters including a body defining an opening for receiving a test strip, an interface port disposed within the body, wherein the interface port is configured to read the signal from the test strip, and an electronic circuit configured to transmit the signal to the adapter port. Further, each of the plurality of adapters has different physical dimensions of the interface port.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### BRIEF DESCRIPTION OF THE DRAWINGS

The related drawings illustrate all the preferred embodiments of the invention wherein:

FIG. 1 illustrates an exploded view of a portable electronic device, according to an embodiment of the present invention;

FIG. 2 illustrates a front view of a portable electronic device, according to an embodiment of the present invention;

FIG. 3 illustrates an exploded view of an adapter for use with a portable electronic device, according to an embodiment of the present invention;

FIG. 4 illustrates a schematic of an adapter, in accordance with an embodiment of the present invention;

FIG. 5 illustrates a schematic of an adapter, in accordance with another embodiment of the present invention;

FIG. 6A illustrates a front perspective view of a portable electronic device coupled to a mobile device, in accordance with an embodiment of the present invention;

FIG. 6B illustrates a rear perspective view of a portable electronic device coupled to a mobile device, in accordance with an embodiment of the present invention;

FIG. 6C illustrates a left side view of a portable electronic device coupled to a mobile device, in accordance with an embodiment of the present invention;

FIG. 6D illustrates a right side view of a portable electronic device coupled to a mobile device, in accordance with an embodiment of the present invention;

FIG. 6E illustrates a bottom view of a portable electronic device coupled to a mobile device, in accordance with an embodiment of the present invention;

FIG. 7 illustrates an adapter being inserted in a slot disposed on a portable electronic device, in accordance with an embodiment of the present invention;

FIG. 8A illustrates a lock ring in a locked state, in accordance with an embodiment of the present invention;

FIG. 8B illustrates a lock ring in an unlocked state, in accordance with an embodiment of the present invention;

FIG. 9 illustrates a portable electronic device with a storage cover removed, in accordance with an embodiment of the present invention;

FIG. 10A-10H illustrate screenshots of a user interface, in accordance with various embodiments of the present invention;

FIG. 11 illustrates a perspective view of a portable electronic device, in accordance with embodiment of the present invention;

FIG. 12A illustrates a front perspective view of a portable electronic device, in accordance with an embodiment of the present invention;

FIG. 12B illustrates a rear perspective view of a portable electronic device, in accordance with an embodiment of the present invention;

FIG. 13A illustrates a front view of a portable electronic device coupled to a case for a mobile device, in accordance with an embodiment of the present invention;

FIG. 13B illustrates a rear view of a portable electronic device coupled to a case for a mobile device, in accordance with an embodiment of the present invention;

FIG. 14 illustrates a side view of a portable electronic device coupled to a mobile device having a case, in accordance with an embodiment of the present invention;

FIG. 15 illustrates a hardware block diagram of a portable electronic device, according to an embodiment of the present invention;

FIG. 16A illustrates an isometric view of a portable electronic device, according to an embodiment of the present invention;

FIG. 16B illustrates a front view of a portable electronic device, according to an embodiment of the present invention;

FIG. 16C illustrates a side view of a portable electronic device, according to an embodiment of the present invention;

FIG. 17A illustrates a cross-sectional view of a portable electronic device, according to an embodiment of the present invention;

FIG. 17B illustrates a cross-sectional view of a portable electronic device, according to an embodiment of the present invention;

FIG. 18A illustrates a hardware block diagram of a portable electronic device, according to an embodiment of the present invention;

FIG. 18B illustrates a hardware block diagram of a portable electronic device, according to an embodiment of the present invention;

FIG. 19 illustrates a block diagram of a circuit board of a portable glucose monitoring device, according to an embodiment of the present invention;

FIG. 20 illustrates a functional block diagram of a portable electronic device, according to an embodiment of the present invention;

FIG. 21 illustrates a flowchart schematically outlining a method for analyzing an analyte using a portable electronic device, according to an embodiment of the present invention; and

FIG. 22 illustrates a chipset upon which an embodiment of the present invention may be implemented.

### DETAILED DESCRIPTION

Embodiments of the present invention will be illustrated below in conjunction with exemplary configurations of portable electronic devices and systems for analyzing an analyte.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The terms “first end,” “second end,” used herein do not denote any order, but rather are used to distinguish one end from another. However, in some embodiments, the first end and the second end can refer to one end.

The term “A1C” used herein, refers to A1C test (also known as HbA1C or glycated hemoglobin) that provides a good general indication of diabetes control. The test is used to indicate a person's average blood glucose level over the past few months.

Embodiments of the present invention include a portable electronic device for analyzing an analyte. The analyte may include glucose, lactate, blood gases (e.g., carbon dioxide or oxygen), blood PH, hemoglobin, or any other biological species present in a biological fluid or sample, such as blood, sweat, urine, plasma, serum and the like.

The portable electronic device includes a housing, an adapter detachably coupled to the housing, and a processor disposed in housing. The adapter includes an opening to receive a test strip. The portable electronic device determines a parameter of the analyte, for example, glucose levels. If glucose is used as the analyte, a lancet is used to prick skin of a patient and one or more drops of the blood are placed on the test strip. The presence of the analyte on the test strip causes an electro-chemical reaction in the test strip. The test strip generates a signal based on the electro-chemical reaction. The test strip is inserted into the portable electronic device. The adapter includes an interface port that reads the signal from the test strip. The processor, being communicably coupled to the interface port, determines glucose levels based on the read signal and transmits them to a mobile device with a display.

In an embodiment, the portable electronic device includes a universal adapter port that can interface with different types of adapters. Each type of adapter is compatible with a specific type of test strip. Dimensions of the interface port of each type of adapter may vary based on the corresponding test strip.

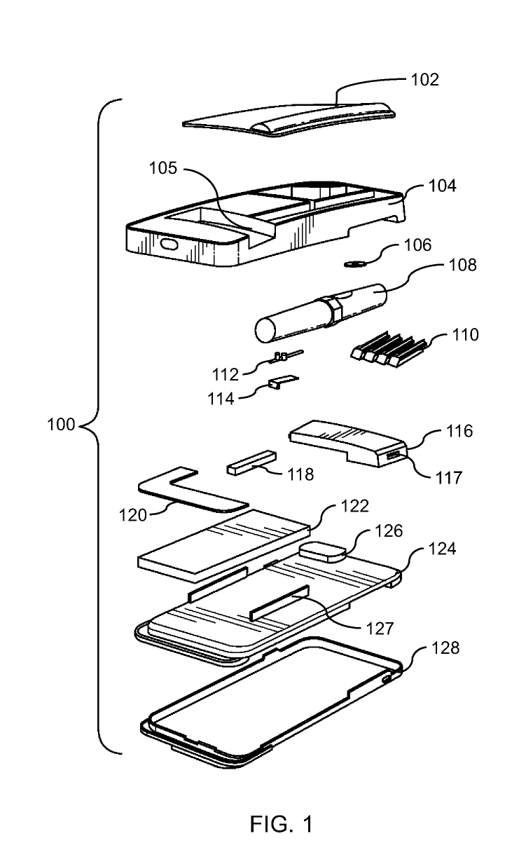

Various embodiments of the present inventions are presented by way of examples illustrated in the FIGS. 1-22.

FIG. 1 illustrates an exploded view of a portable electronic device 100. In an embodiment, the portable electronic device 100 is utilized for monitoring glucose levels in a blood sample. The portable electronic device 100 includes a storage cover 102 covering a housing 104 with multiple compartments. The storage cover 102 is detachably coupled to the housing 104 using a lock ring 106, a lock pin 112 and a lock bracket 114. The multiple compartments of the housing 104 stores at least one lancet 108, one or more lancet needles 110 and at least one test strip 118. The housing 104 further includes a slot 105 to position an adapter 116. The adapter 116 is positioned in the slot 105, such that the adapter 116 is connected to a printed circuit board (PCB) 120 through an adapter port. The PCB 120 includes a processor (not shown) that may be, but not restricted to, a Central Processing Unit (CPU), a microprocessor, or a microcontroller for calculating and transmitting data obtained by measuring blood glucose levels.

The lancet 108 is used to prick the skin of a patient to obtain droplets of blood. The droplets of blood are placed on the test strip 118. The test strip 118 is inserted into the adapter 116. When the blood is placed onto the test strip 118, a signal is generated by a chemical reaction caused by an interface of glucose present in the patient's blood, and a chemically treated metal (not shown) on the test strip 118.

The adapter 116 includes a body with an opening 117. The adapter 116 receives the test strip 118 through the opening 117. The adapter 116 further includes an interface port (not shown in FIG. 1) that is disposed within the body. The interface port reads a signal generated by the test strip 118. The signal may be an electric signal or a magnetic signal. In an embodiment, the signal may be an electric current or a voltage.

In some embodiments, the test strip 118 is a composite film that is a combination of adhesive materials and an electronic circuit. The test strip 118 includes a sample chamber (not shown) that induces rapid blood absorption. In some embodiments, the test strip may include visual cues, such as a change in color, to indicate that sufficient blood to generate a signal is placed on the test strip 118. The test strip 118 further includes an enzyme, such as glucose oxidase that electrochemically reacts with the blood, so that a signal is generated. Electrons from glucose travel through a network of wires in the test strip 118, thereby generating current. When the test strip 118 is inserted into the adapter 116 through the opening 117, and the adapter 116 is inserted into the slot 105, the interface port of the adapter 116 reads the current. The PCB 120 counts the electrons as current and determines the amount of glucose needed to generate the current.

In some embodiments, the adapter 116 is selected from multiple adapters. Each of the multiple adapters includes an interface port. Each of the multiple adapters is configured to interface with a corresponding type of test strip. The housing 104 may store different types of test strips. Test strips may differ on the basis of materials used, shape, dimensions and the like. Further, different test strips may correspond to different manufacturers. Each of the multiple adapters can be inserted into the slot, such that each adapter is connected to the PCB 120 through the adapter port.

The adapter 116 and the PCB 120 are powered by a battery 122 (shown in FIG. 1) placed in a battery holder 127. The battery holder 127 is disposed on a case body 124. Further, the housing 104 is attached to the case body 124. The case body 124 is further attached to a frame 128. In an embodiment, the frame 128 may be a rectangular frame that provides support to the housing 104 and the case body 124. The frame 128 is further attached to a mobile device, such as, but not limited to a phone, a tablet computer and the like. In some embodiments, the frame 128 may be detachably coupled to a mobile device using a clip (not shown).

In some embodiments, the case body 124 and the housing 104 may include openings to position external buttons and/or external devices of the mobile device to which the case body 124 and the housing 104 are attached using the frame 128. For example, the case body 124, the housing 104 and the frame 128 may be attached to a smartphone that includes a camera. Accordingly, the case body 124 is provided with an opening 126 that is positioned above the camera of the phone. Further the housing 104 may also include an opening aligned with the opening 126 of the case body 124. Other openings on the housing 104 may include openings for protruding volume buttons and power buttons of the attached smartphone.

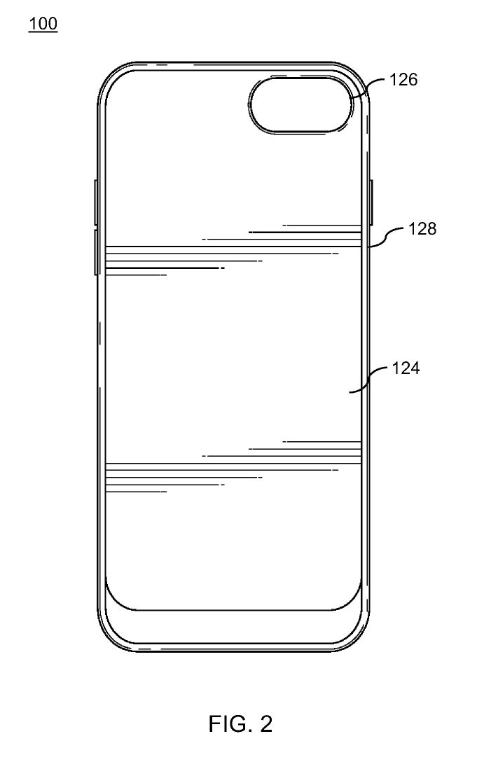

FIG. 2 illustrates a front view of the portable electronic device 100. The case body 124 is fixed to the frame 128 is shown. A mobile device of similar dimensions as the case body 124 may be attached, such that any camera or protruding buttons may coincide with the opening 126.

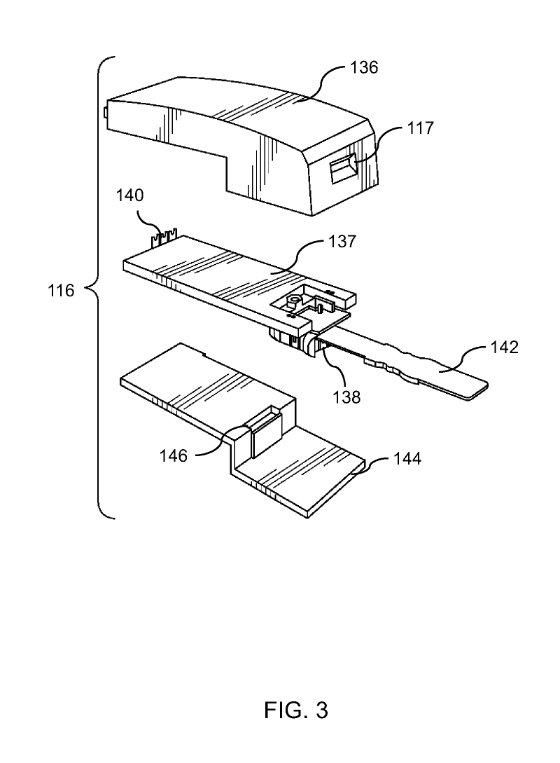

FIG. 3 illustrates an exploded view of the adapter 116, in accordance with some embodiments of the present invention. The adapter 116 includes a body 136 with an opening 117. The adapter 116 further includes a PCB 137 with an interface port 138 and connection terminals 140. The PCB 137 includes an electronic circuit. In some embodiments, the connection terminals 140 may be strips of a conducting material. The interface port 138 reads a signal generated by a test strip 142. The signal may be an electric signal or a magnetic signal. In FIG. 2, the PCB 137 may be attached to the body 136. The interface port 138 receives the test strip 142 through the opening 117. The adapter 116 further includes an adapter bottom 144 with a clip 146. The adapter body 136 and the PCB 137 are connected to the adapter bottom 144 using the clip 146. In some embodiments, the clip 146 may be part of a latching mechanism. The adapter 116 is detachably coupled to the slot 105 of the housing 104 (shown in FIG. 1), such that the connection terminals 140 contact an adapter port that is communicably coupled to the PCB 120 disposed in the housing 104.

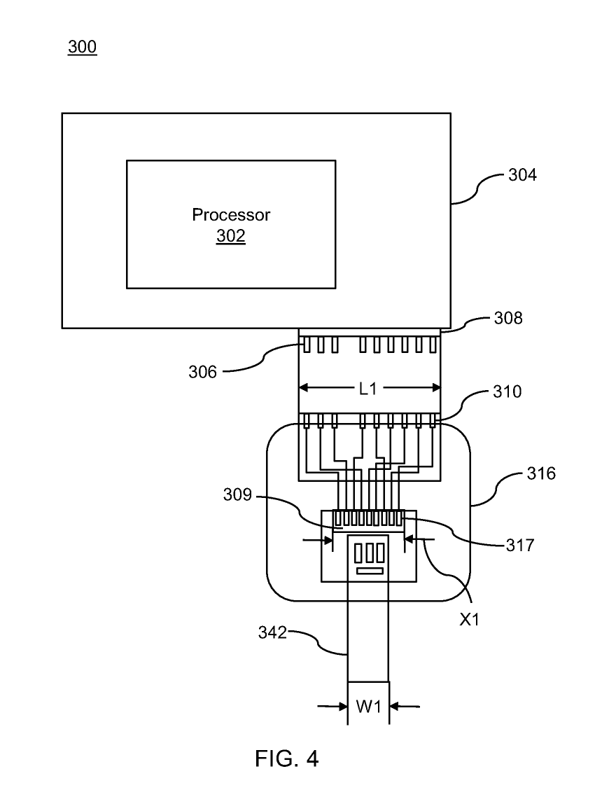

FIG. 4 illustrates a schematic 300 of an adapter 316 with an inserted test strip 342. The test strip has a width ‘W1’. The adapter 316 is connected to a PCB 304 through an adapter port 308 of length ‘L1’. The PCB 304 includes a processor 302 that may be, but not restricted to, a Central Processing Unit (CPU), microprocessor, or a microcontroller. The PCB 304 may also include a memory, input/output ports, a clock, and the like. The test strip 342 is inserted through an opening of the adapter 316. The test strip 342 is positioned such that the test strip 342 contacts contact terminals 317 of an interface port 309. In some embodiments, the contact terminals 317 may be strips of a conducting material. The interface port 309 has a width ‘X1’. In an embodiment, the width ‘X1’ of the interface port 309 may be substantially equal to the width ‘W1’ of the test strip 342. The contact terminals 317 are electrically coupled to connection terminals 310. The connection terminals 310 extend over the length ‘L1’ of the adapter port 308. The adapter port 308 includes adapter terminals 306 that contact the connection terminals 310 when the adapter 316 is coupled to the adapter port 308. The test strip 342 generates a signal that is transmitted to the connection terminals 310 when the test strip 342 is inserted into the adapter 316 and the adapter 316 is coupled to the adapter port 308. The adapter 316 may include an electronic circuit (not shown) that transmits the signal to the PCB 304 through the adapter port 308. The PCB 304 determines one or more parameters, for example, glucose levels, from the transmitted signal.

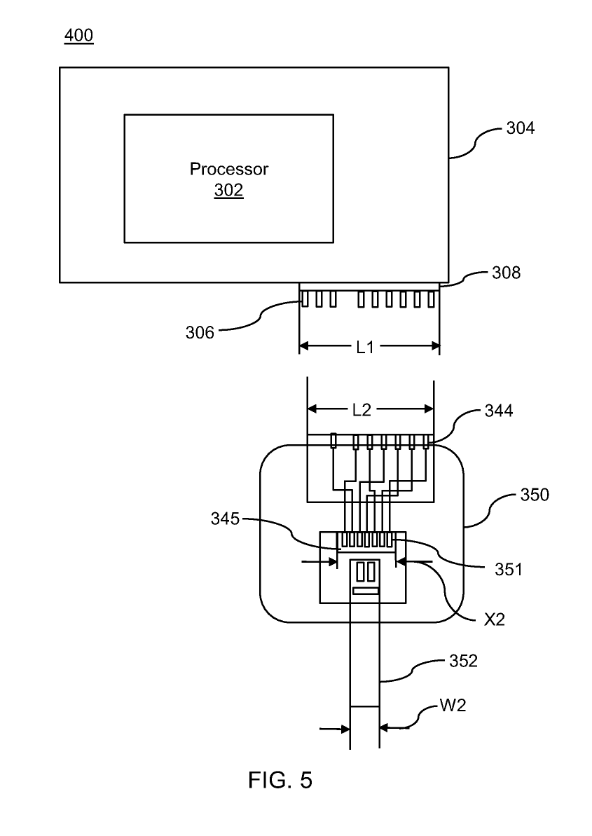

FIG. 5 illustrates a schematic 400 of an adapter 350 with an inserted test strip 352. The test strip 352 has a width ‘W2’. In an embodiment, the width ‘W2’ of the test strip 352 is lesser than the width ‘W1’ of the test strip 342 illustrated in FIG. 4. The adapter 350 is connected to a PCB 304 through an adapter port 308 of length ‘L1’. The test strip 342 is inserted through an opening of the adapter 350. The test strip 342 is positioned within the opening, such that the test strip 342 contacts contact terminals 351 of an interface port 345. The interface port 345 has a width ‘X2’. In some embodiments, the contact terminals 351 may be strips of a conducting material. The contact terminals 351 are electrically coupled to connection terminals 344. The connection terminals 344 extend over a length ‘L2’ of the adapter port 308. The length ‘L1’ is greater than the length ‘L2’. In an embodiment, the width ‘X2’ of the interface port 345 may be substantially equal to the width ‘W2’ of the test strip 352. Further, the width ‘X2’ of interface port 345 is lesser than the width ‘X1’ of the interface port 309 illustrated in FIG. 4. Therefore, interface ports of adapters have varying dimensions based on the corresponding type of test strip. Differences in dimensions may further include differences in lengths, thicknesses and shapes in addition to difference in widths.

Further, the adapter port 308 includes adapter terminals 306 that contact the connection terminals 344 when the adapter 350 is coupled to the adapter port 308. The test strip 352 generates a signal that is transmitted to the connection terminals 344 when the test strip 352 is inserted into the adapter 350 and the adapter 350 is coupled to the adapter port 308. The adapter 350 may include an electronic circuit (not shown) that transmits the signal to the PCB 304 through the adapter port 308. The PCB 304 determines one or more parameters, such as glucose levels, from the transmitted signal.

The adapter port 308 is compatible with adapter ports 316 and 350, in spite of varying dimensions. One of the adapters 316 and 350 may be selected to measure glucose levels based on the type of test strip. The adapter 316 is used if the test strip 342 is utilized for collecting blood or an analyte. Further, the adapter 350 is used if the test strip 352 is utilized for collecting blood or an analyte.

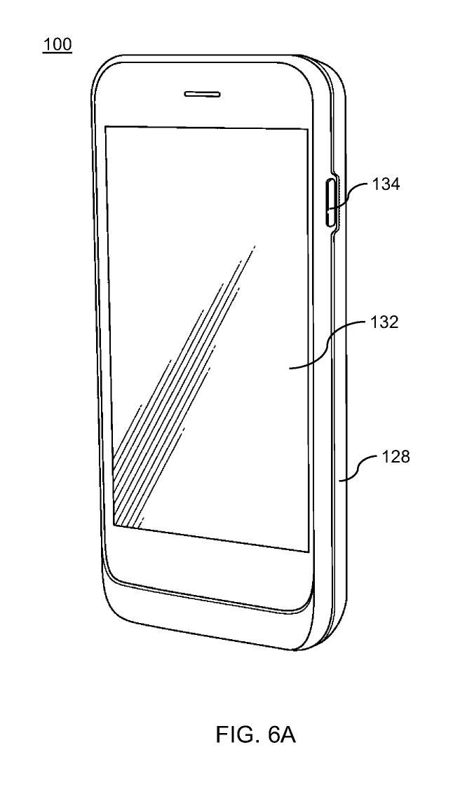

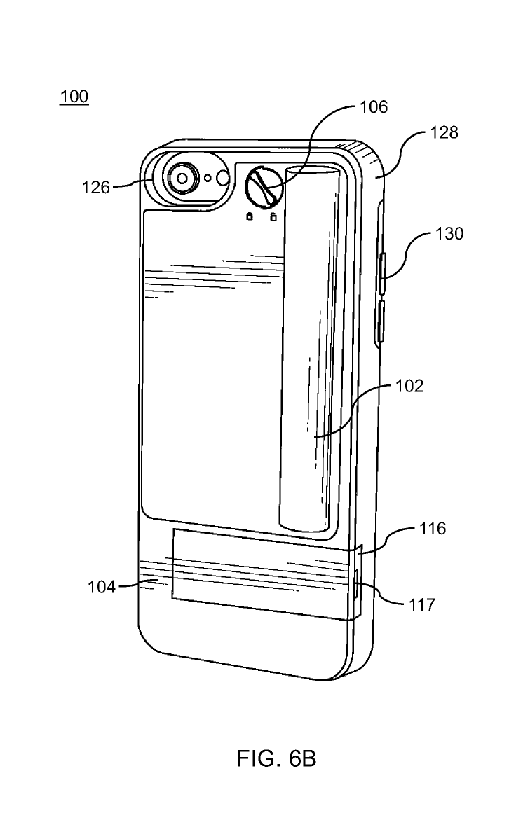

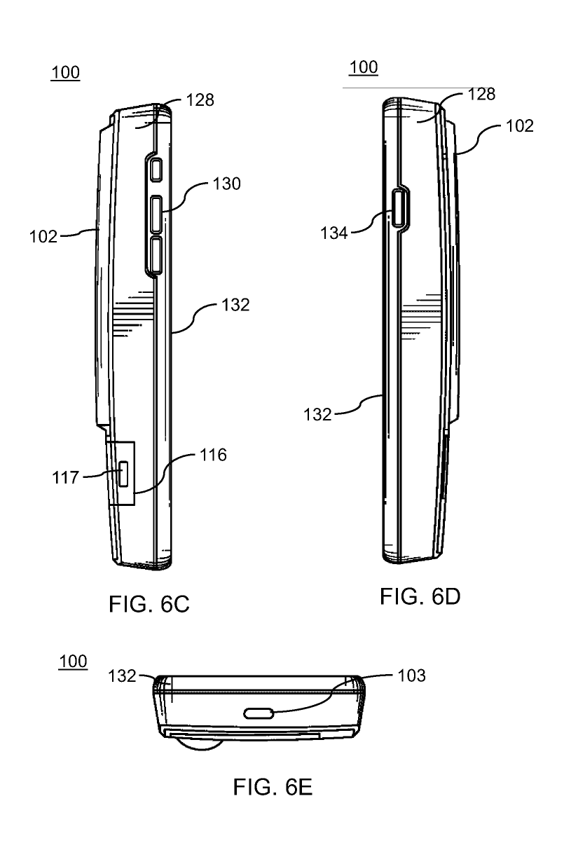

FIG. 6A illustrates a perspective view of the portable electronic device 100 (shown in FIG. 1) with a mobile device 132 attached to the frame 128. The mobile device 132 may be, but not limited to a mobile phone, a tablet computer, an external display and the like. The mobile device 132 is attached to the frame 128 such that one or more buttons on the mobile phone may coincide with an opening 134 on the frame 128. In some embodiments, the phone grip may include external button covers which coincide with corresponding buttons of the mobile device 132.

FIG. 6B illustrates a rear view of the portable electronic device 100 coupled to the mobile device 132 (shown in FIG. 6A). The mobile device 132 is detachably coupled to the portable electronic device 100 such that one or more buttons on the mobile device 132 coincide with an opening 130 on the frame 128, and a camera lens protruding from the mobile device 132 coincides with the opening 126. The storage cover 102 is shown detachably coupled to the housing 104 using the lock ring 106. FIG. 6B further illustrates the adapter 116 coupled to the portable electronic device 100. The adapter 116 includes the opening 117 for a test strip to be inserted.

FIGS. 6C and 6D illustrate side views of the portable electronic device 100 coupled to the mobile device 132, such that buttons of the mobile device 132 coincide with the openings 130 and 134. The portable electronic device 100 includes the storage cover 102, the adapter 116 and the frame 128. FIG. 6E is a bottom view of the portable electronic device 100. The portable electronic device 100 includes a charging port 103 to which an external power source may be coupled. In some embodiments, the mobile device 132 may be electrically coupled to the portable electronic device 100 through the charging port 103. The charging port 103 may include interfaces, such as, but not limiting to, Universal Serial Bus (USB), USB-C, lightening connector, micro-USB and the like.

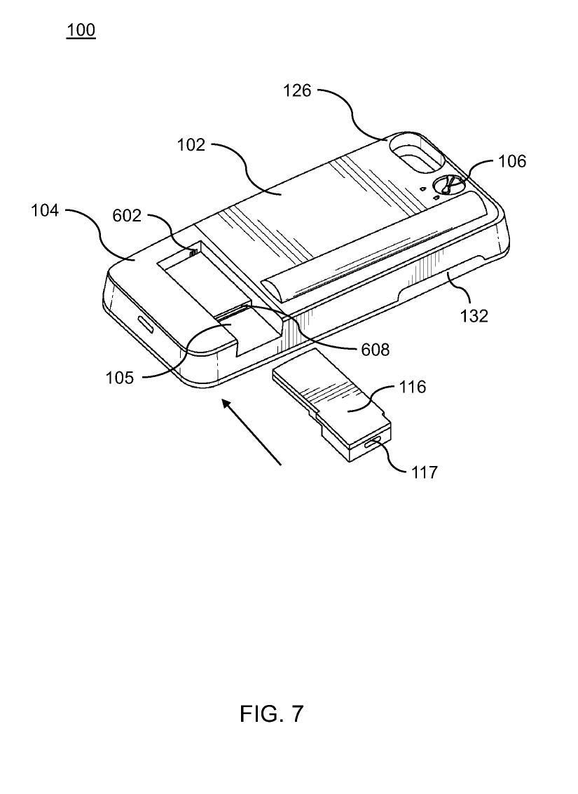

FIG. 7 illustrates the adapter 116 being inserted in the slot 105 of the portable electronic device 100. The portable electronic device 100 includes the storage cover 102 and the housing 104. The lock ring 106 detachably couples the storage cover 102 to the housing 104. The portable electronic device 100 may be coupled to the mobile device 132, such that a camera lens of the mobile device 132 may coincide with the opening 126. The adapter 116 with the opening 117 is inserted in the slot 105 such that the adapter 116 is latched to an opening 608. Once latched, the adapter 116 is in contact with an adapter port 602. In another embodiment, the adapter 116 may be detachably coupled to the slot 105 by a snap-fit mechanism. The adapter 116 is connected to the PCB 120 (shown in FIG. 1) through the adapter port 602. The adapter 116 is selected from multiple adapters, each adapter being compatible with a corresponding test strip. A compatible test strip placed with droplets of blood is inserted in the opening 117.

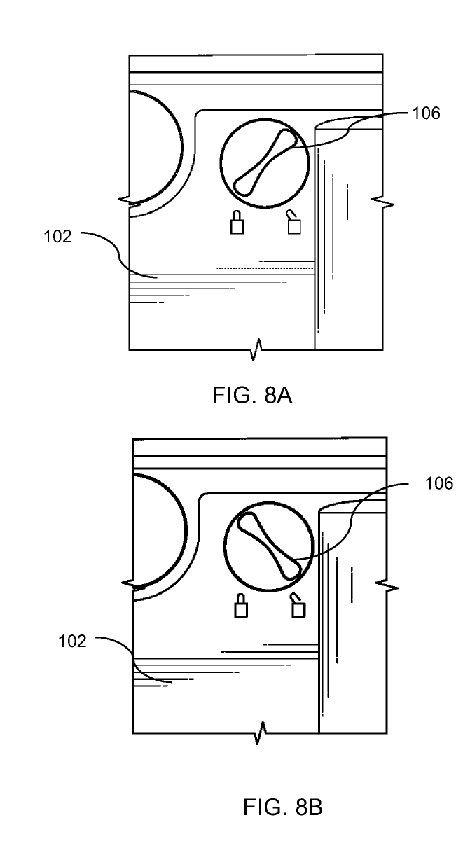

FIGS. 8A and 8B illustrate the lock ring 106. A user may use the lock ring 106 to couple the storage cover 102 to the housing 104 or remove the storage cover 102 from the housing 104. The lock ring 106 is a knob that locks the storage cover 102. In FIG. 8A, the lock ring 106 is in a locking position and couples the storage cover 102 to the housing 104. In FIG. 8B, the lock ring 106 may be rotated to an unlocking position, such that the storage cover 102 may be removed. In an embodiment, the storage cover 102 may include indicia to indicate the locking and unlocking positions of the storage cover 102. Upon releasing the storage cover 102, the user may utilize the lancet 108 (shown in FIG. 1) and one of the lancet needles 110 (shown in FIG. 1) stored in multiple compartments of the housing 104 to extract a few drops of blood.

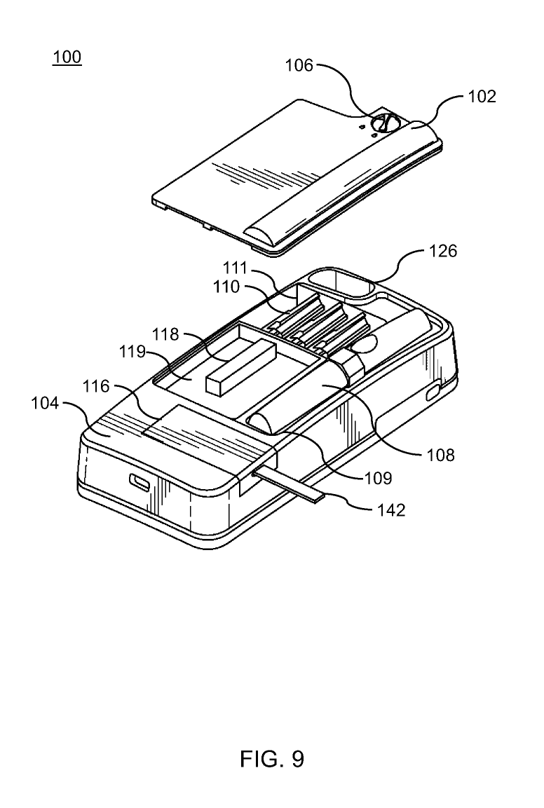

FIG. 9 illustrates the portable electronic device 100 with the storage cover 102 removed from the housing 104. As shown in FIG. 9, the housing 104 includes compartments 111, 119 and 109. The user fixes one of the lancet needles 110 to the lancet 108 to prick his/her skin to extract a one or more drops of blood. The lancet needles are stored in the compartment 111 and the lancet 108 is stored in the compartment 109. In some embodiments, the housing 104 may include a lancet slider (not shown), a lancet port (not shown) and a lancet trigger/release button (not shown). In another embodiment of the present invention, the housing 104 may include a compartment to accept a separate lancet device containing a lancet slider (not shown), a lancet port (not shown) and a lancet trigger/release button (not shown). The lancet slider slides the lancet through the lancet port. The lancet slider may be used to adjust lancing tension. The lancet trigger/release button is pressed to trigger the lancet for piercing the skin and release the lancet after piercing the skin of the user. One or more drops of blood are extracted and placed on the test strip 142. The test strip 142 may be the at least one test strip 118 stored in the compartment 119. The test strip 142 is inserted in the adapter 116 through the opening 117. The test strip 142 generates a signal based on an electrochemical reaction between the drops of blood and the test strip. The interface port 138 (shown in FIG. 3) of the adapter 116 reads the signal. The adapter 116 transmits the signal to the PCB 120 through the adapter port 308, when the adapter 116 is electrically coupled to the PCB 120 (shown in FIG. 1) disposed in the housing 104. The processor 302 on the PCB 304 calculates one or more parameters pertaining to glucose levels in the drops of blood placed on the test strip 142.

In some embodiments, the portable electronic device 100 may be communicably coupled to the mobile device 132 through a communication port. The communication port may include interfaces, but not limiting to, Universal Serial Bus (USB), USB-C, lightening connector, micro-USB and the like. In other embodiments, the portable electronic device 100 may be communicably coupled to the mobile device 132 through communication interfaces, such as Bluetooth™, near field communication, ISM, Bluetooth™ Low Energy (BLE), ZigBee, WLAN standard or over the Internet. The processor 302 is configured to generate a user interface on a display of the mobile device 132 coupled to the portable electronic device 100. The processor 302 may execute instructions to generate the user interface and display indicia indicative of the one or more parameters pertaining to glucose levels. The mobile device 132 may also execute a software application for displaying the user interface. In an alternative, the portable electronic device 100 may include an onboard display (not shown) for displaying the user interface.

In some embodiments, the processor 302 may generate indicia indicative of the one or more parameters and transmit the indicia to a server (not shown) via a communication network. The communication network may be, but not limited to, a local area network (LAN), a Wide Area Network (WAN) or any wireless network. The server may then transmit the one or more parameters to the mobile device 132. The mobile device 132 may display the one or parameters through a user interface on the mobile device 132.

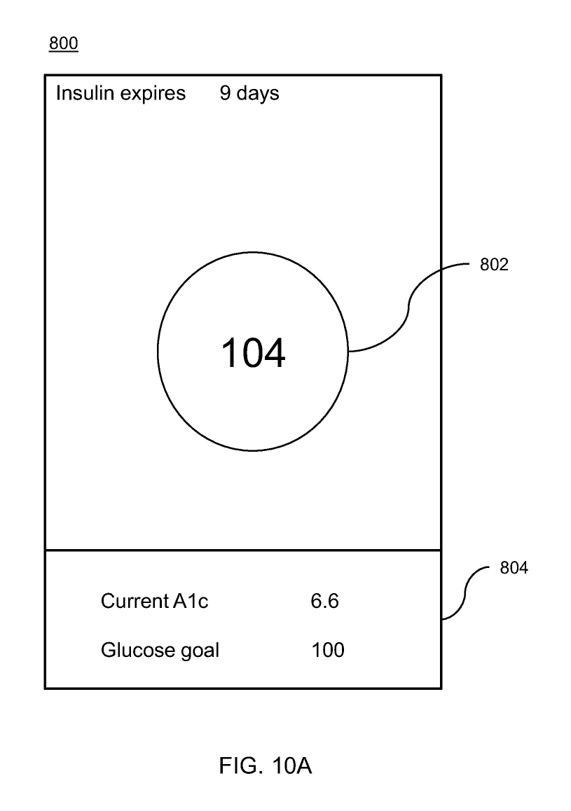

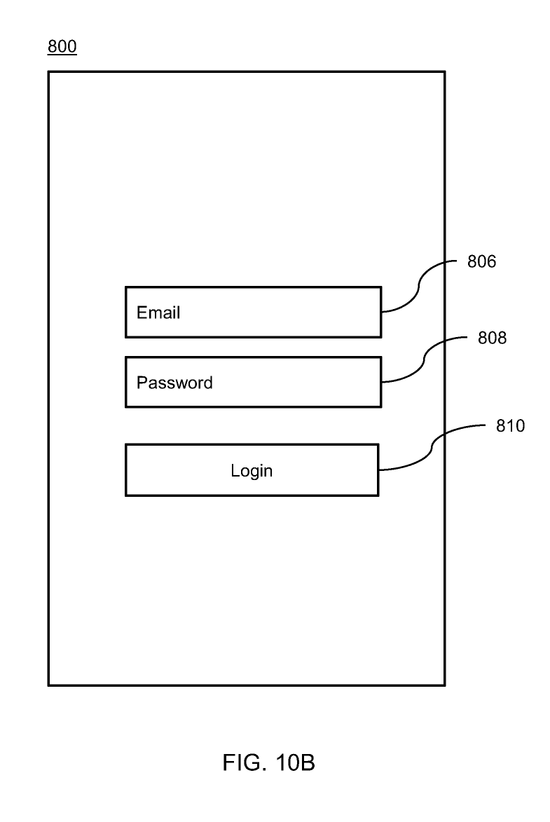

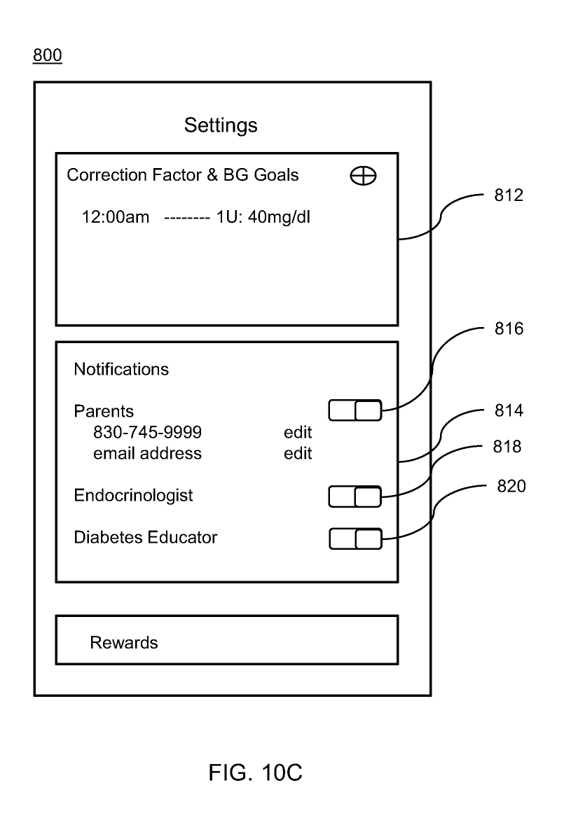

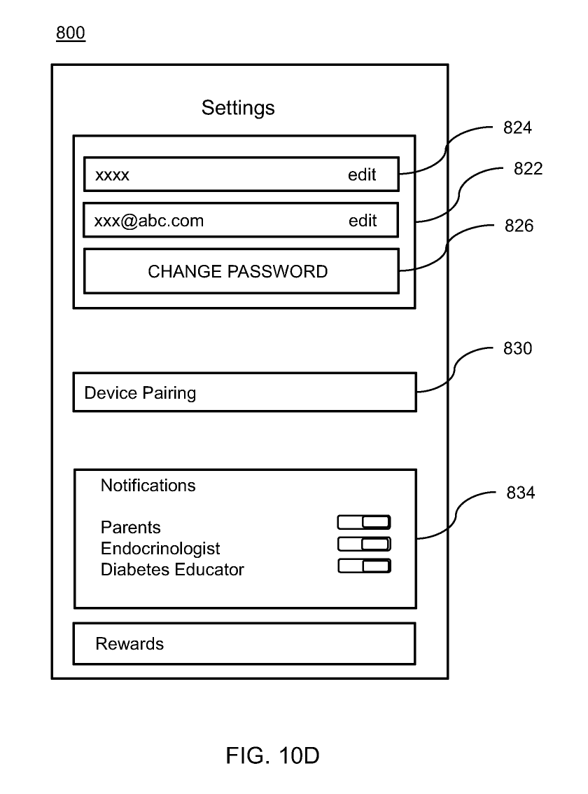

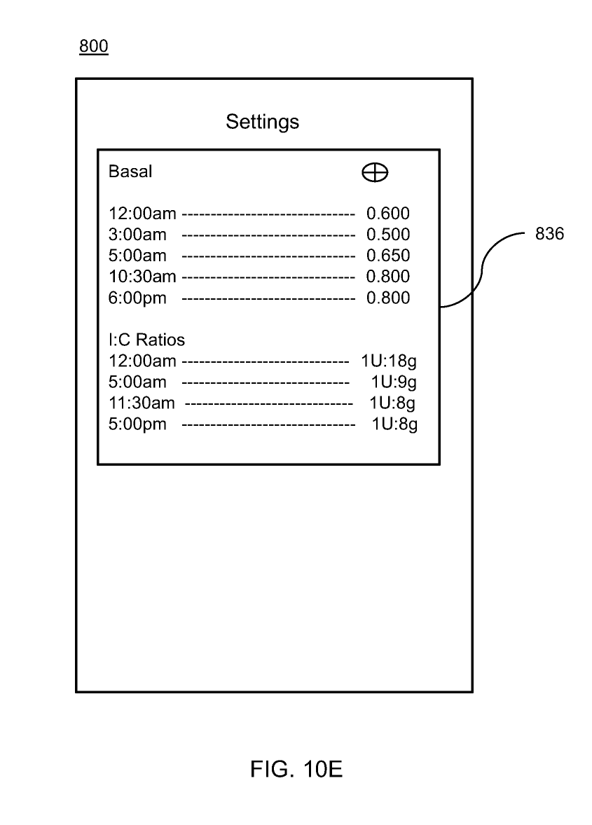

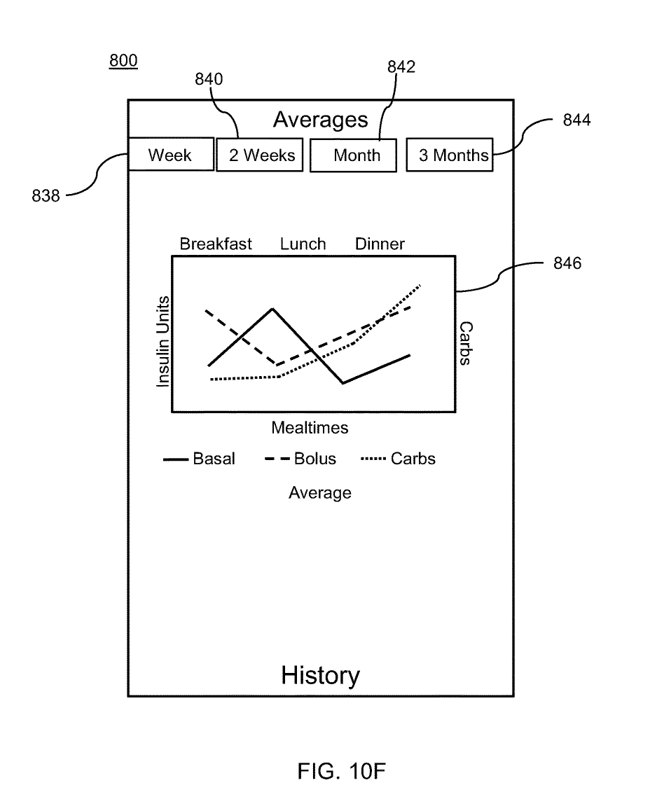

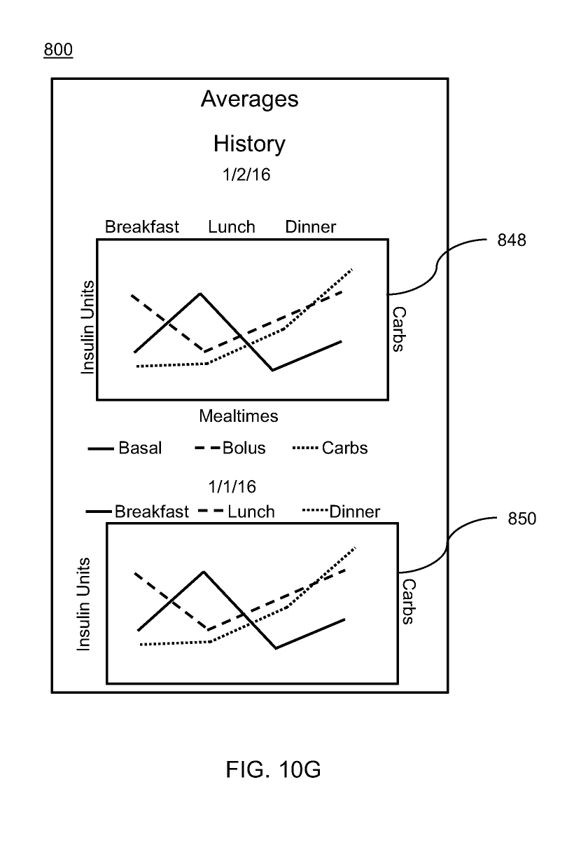

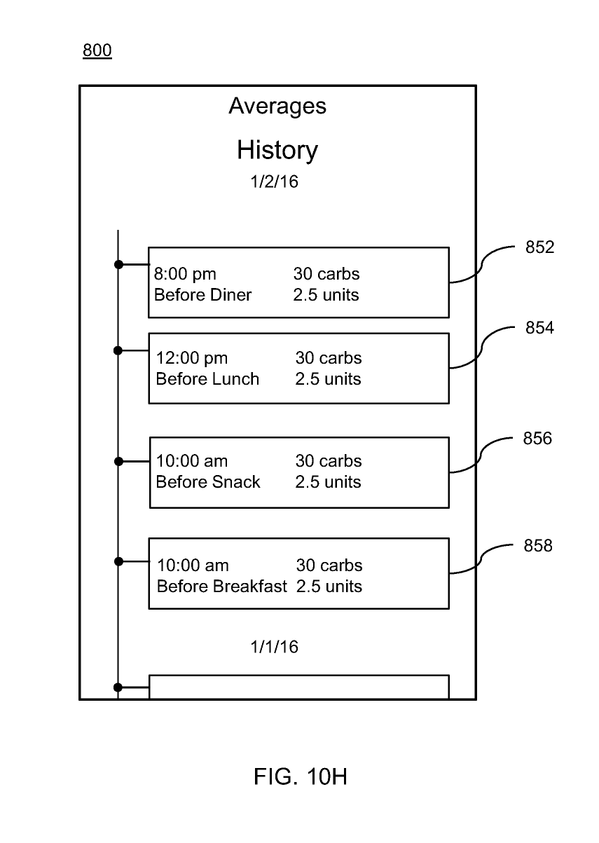

FIGS. 10A to 10H illustrate a user interface 800 that displays indicia generated by the processor 302. The user interface 800 may be displayed on the mobile device 132. In FIG. 10A, the user interface 800 shows the glucose level 802 and metrics pertaining to A1c levels and future goals of the user. The user may also be directed to register an account with a health management system. Further, as shown in FIG. 10B, a user may be directed to authenticate registration details by providing an email address 806 and a password 808 on the user interface 800. Upon clicking a login button 810, the user is allowed to access his/her account. FIG. 10C illustrates the user interface 900 displaying settings pertaining to the registered account of the user. The settings include a box 812 to set health goals. Further the settings include options 814 to send notifications to a parent or a guardian, an endocrinologist, and a diabetes educator by enabling slide buttons 816, 818 and 820.

FIG. 10D illustrates the user interface 800 displaying further details pertaining to the authenticated account. The settings include a name field 824 and an email address field 822. Password to the account may be change by clicking a button 826. The user interface 800 provides options to pair the mobile device 132 with an external device such as an insulin pump through Bluetooth™ or any shortwave communication. In an example, the mobile device 132 may be paired with an insulin pump. The data obtained from the processor 302 is used to control settings and distribution of insulin from the insulin pump either manually or transmitted via shortwave communication directly to the insulin pump. The mobile device 132 is paired with an external device upon clicking a button 830. In some embodiments, the user may be directed a list of devices that may be paired with the mobile device 132. Any notifications to be transmitted to parents, an endocrinologist or a diabetes educator are enabled at a field box 834.

In FIG. 10E, historical medical data such as basal metabolic rates and insulin to carbohydrate ratios are displayed at a field box 836. In FIG. 10F, a graph 846, pertaining to average insulin dosages and average insulin to carbohydrate ratios, is represented on the user interface 800. Graphical data with respect to a week, a fortnight, a month and three months may be displayed upon clicking the buttons 838, 840, 842 and 844 respectively.

In FIG. 10G, a graph 848 displays insulin dosages and insulin to carbohydrate ratios for specific dates. The graph 848 represents data tabulated on 2 Jan. 2016 and the graph 850 represents data tabulated on 1 Jan. 2016. In FIG. 10H, a medical history of the user is displayed. Field boxes 852, 854, 856 and 858 display insulin to carbohydrate details at different times during a day.

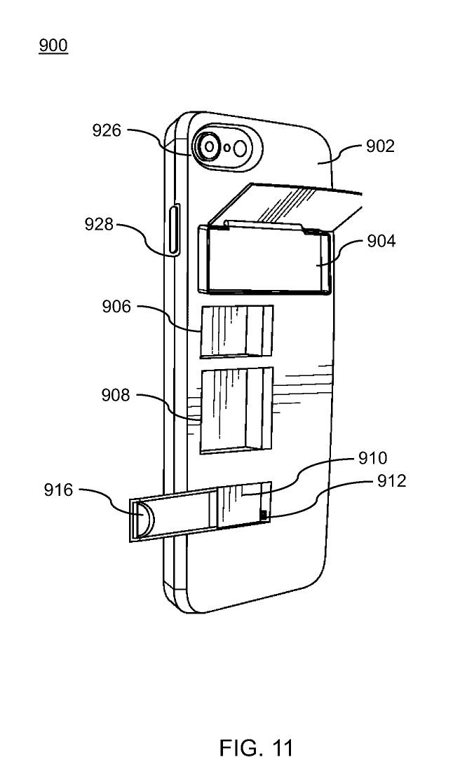

FIG. 11 illustrates an exemplary embodiment of a portable electronic device 900 for analyzing an analyte. The portable electronic device 900 includes a housing 902 with compartments 904, 906 and 908 storing a lancet (not shown), lancet needles (not shown) and test strips (not shown), respectively. The portable electronic device 900 further includes an adapter 916 that is inserted through a slot 910. The adapter 916 is positioned in the slot 910 such that it is in contact with an adapter port 912. The housing 902 is detachably coupled to a mobile device such that any external buttons on the mobile device may coincide with an opening 928. Further, the mobile device is detachably coupled to the housing 900, such that any camera lens on the mobile device may coincide with an opening 926. A user extracts drops of blood by pricking his/her skin using a lancet and a lancet needle (not shown). The lancet and lancet needle may be stored in the compartments 904 and 906, respectively. The drops of blood are placed on a test strip (not shown). The test strip may be stored in the compartment 908. The test strip is inserted in the adapter 916. When the adapter 916 is inserted through the slot 912 and is in contact with the adapter port 912, the test strip, the adapter 916 and the adapter port 912 are electrically coupled. The adapter port reads a signal generated by the test strip. The signal is generated through an electrochemical reaction between the blood and the test strip. The signal is transmitted to a PCB (not shown) disposed within the housing 902. The PCB includes a processor that calculates parameters indicative of a glucose level of the blood. The processor may transmit the parameters to the mobile device. A user may access the parameters and any derived information from through a user interface displayed on the mobile device.

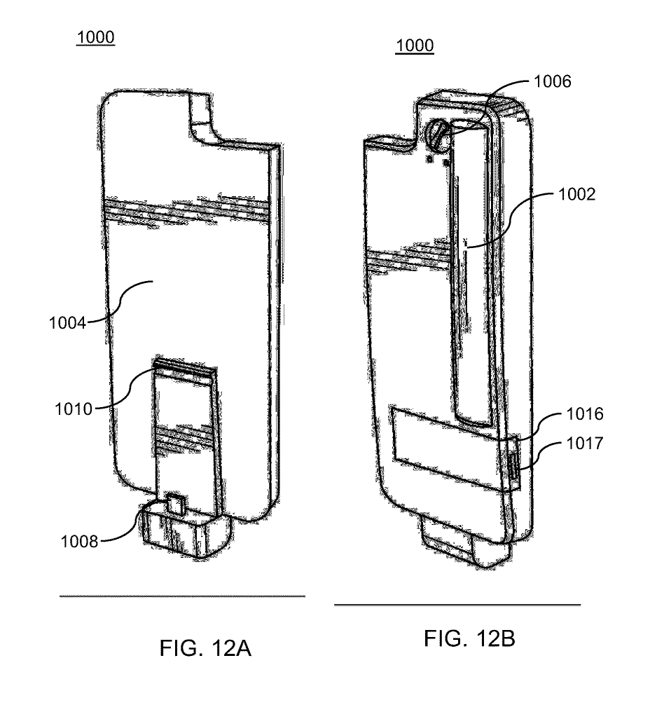

FIG. 12A illustrates a front view of a portable electronic device 1000 for analyzing an analyte. The portable electronic device 1000 includes a housing 1004 with a clip 1010 and a communication port 1008.

FIG. 12B illustrates a rear view of the portable electronic device 1000. The portable electronic device 1000 includes a storage cover 1002 detachably coupled to the housing using a lock ring 1006. The portable electronic device 1000 further includes an adapter 1016 that is detachably inserted through a slot disposed on the housing 1004. The adapter 1016 is positioned in the slot such that the adapter 1016 is in contact with an adapter port (not shown) disposed in the slot. A PCB (not shown), disposed in the housing 1004, is coupled to the adapter 1016 via the adapter port.

A case for a mobile device is detachably coupled to the portable electronic device 1000 using the clip 1010. A mobile device with a case is further communicably coupled to the portable electronic device 1000 by connecting the communication port 1008 to a communication port disposed on the mobile device. The communication port 1008 may include interfaces, such as, but not limiting to, Universal Serial Bus (USB), USB-C, lightening connector, micro-USB and the like.

A user extracts drops of blood by pricking his/her skin using a lancet and a lancet needle (not shown). The drops of blood are placed on a test strip (not shown). The adapter 1016 has an opening 1017 through which a test strip is inserted. The adapter port reads a signal generated by the test strip. The signal is generated through an electrochemical reaction between the blood and the test strip. The signal is transmitted to the PCB. The PCB includes a processor that calculates parameters indicative of a glucose level of the blood. The processor may transmit the parameters to the mobile device through the communication port 1008. A user may access the parameters and any derived information through a user interface displayed on the mobile device.

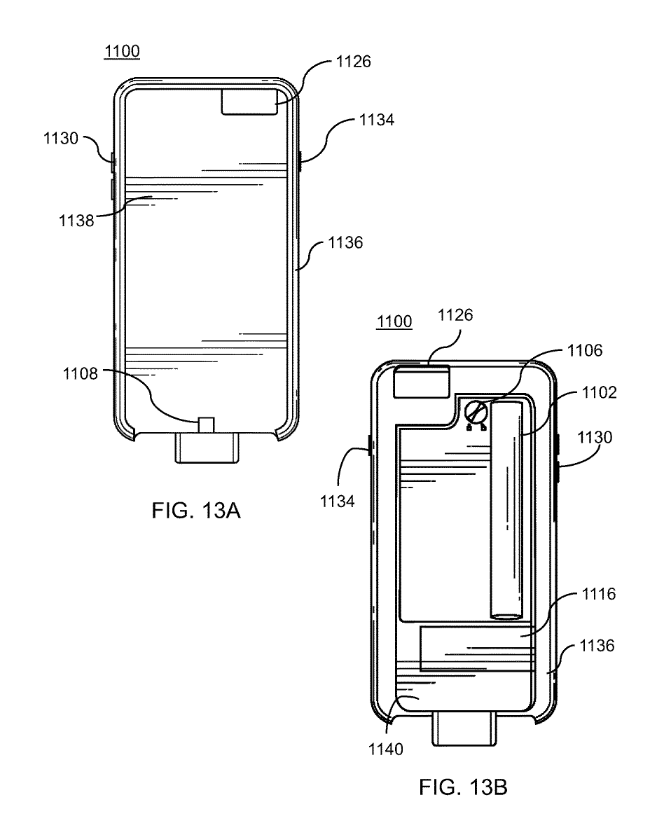

FIG. 13A illustrates a front view of a system 1100 for analyzing an analyte. The system 1100 includes a portable electronic device 1136 coupled to a case 1138 for a mobile device. In some embodiments, the portable electronic device 1136 may be coupled to the case 1138 using a clip. The case 1138 includes external button covers 1130 and 1134. The case 1138 further includes an opening 1126. A mobile device may be coupled to the system 100 by positioning external buttons of the mobile device with the external button covers 1130 and 1134. The mobile device is also positioned, such that a camera lens disposed on the mobile device coincides with the opening 1126. A communication port of the mobile may be coupled to a communication port 1108 of the portable electronic device 1136.

A user extracts drops of blood by pricking his/her skin using a lancet and a lancet needle (not shown). The drops of blood are placed on a test strip (not shown). The adapter 1116 has an opening through which a test strip is inserted. The adapter port reads a signal generated by the test strip. The signal is generated through an electrochemical reaction between the blood and the test strip. The signal is transmitted to the PCB. The PCB includes a processor that calculates parameters indicative of a glucose level of the blood. The processor may transmit the parameters to the mobile device through the communication port 1008. A user may access the parameters and any derived information through a user interface displayed on the mobile device.

FIG. 13B illustrates a rear view of the system 1100. The portable electronic device 1136 includes a housing 1140 and a storage cover 1102. A lock ring 1106 detachably couples the storage cover 1102 to the housing 1140. In some embodiments, the housing 1140 includes one or more compartments underneath the storage cover 1102. Lancets, lancet needles and test strips may be stored in the one or more compartments. The housing 1140 further includes an adapter 1016 that is detachably inserted into a slot. The slot is disposed on the housing 1140. The adapter 1116 is positioned in the slot such that it is in contact with an adapter port (not shown) disposed in the slot. A PCB (not shown) disposed in the housing 1004 is coupled to the adapter 1016 via the adapter port.

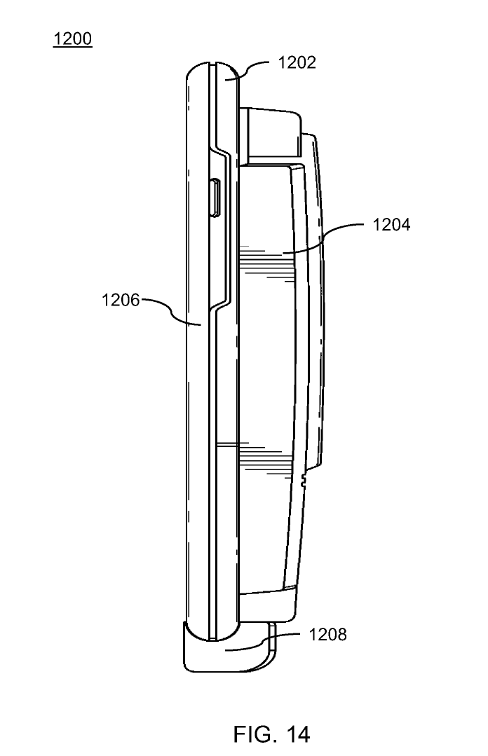

FIG. 14 illustrates a side view of a system 1200 for analyzing an analyte. The system 1200 includes a portable electronic device 1204 coupled to a case 1202 for a mobile device 1206. In some embodiments, the portable electronic device 1204 may be detachably coupled to the case 1202 using a clip. The mobile device 1206 is further coupled to the case 1202. The mobile device is communicably coupled to the portable electronic device 1204 through a communication port 1208.

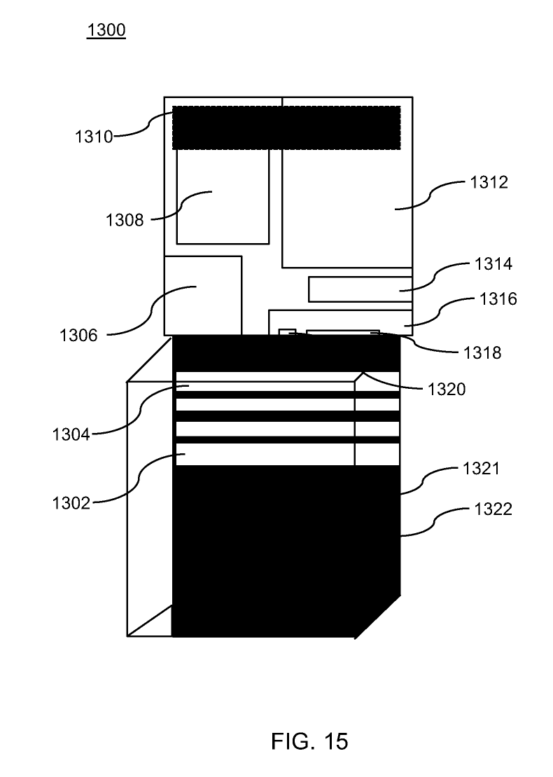

FIG. 15 illustrates a hardware block diagram of a portable electronic device 1300 for analyzing an analyte, according to an embodiment of the present invention. Analyzing an analyte includes measuring an amount of glucose in blood. The portable electronic device 1300 includes a lancet slider 1318 mechanically engaged with a lancet mechanism (not shown) of a lancet port 1316 to slide a lancet through a sliding motion of the lancet slider 1318. The portable electronic device 1300 further includes a lancet trigger/release button 1320 to discharge the lancet. In some embodiments, there can be multiple settings associated with the lancet slider 1318. The lancet pierces the skin of the user based on the pressure applied by the user as per the setting of the lancet slider 1318. The user would then press the lancet trigger/release button 1320 to discharge the lancet. A small amount of blood, released due to piercing, is obtained and put onto a test strip. The test strip is inserted through an interface port 1306.

The interface port 1306 may be of variable sizes having varying widths and depths to accommodate multiple test strips from different manufacturers. The test strip can be any test or sensor strip that analyzes an analyte to determine one or more parameters, such as glucose levels.

When the blood is placed onto the test strip, a signal is generated by a chemical reaction caused between glucose in the blood, and a chemically treated metal on the test strip. A processor 1308 then reads the signal and calculates the amount of glucose in the blood. The processor 1308 may include, but is not restricted to, a Central Processing Unit (CPU), microprocessor, or a microcontroller for calculating and transmitting data obtained by measuring the amount of glucose.

The data obtained by measuring the blood glucose levels is then displayed on a display 1310. The display 1310 may include, but not restricted to, a LCD display, a LED display or any other electronic display capable of displaying measurement results of blood glucose levels.

In some embodiments of the present invention, the data obtained by measuring the amount of glucose is transmitted to other electronic devices including, but not restricted to, a computer, tablet or a cell phone via short range wireless communication. In some embodiments, the electronic devices may be, but not restricted to, cellular phones, Personal Digital Assistants (PDAs), tablet mobile device version, and so forth. In an exemplary scenario, the data obtained by measuring the blood glucose level is transmitted to a mobile phone via Bluetooth™ or any short wave communication signal. In some embodiments of the present invention, the data transferred to the electronic device may be presented in an application installed in the electronic device.

In some embodiments of the present invention, the portable electronic device 1300 is associated with an insulin pump. The data obtained by measuring the blood glucose level is used to control settings and distribution of insulin from the insulin pump either manually or transmitted via shortwave communication directly to the insulin pump.

The portable electronic device 1300 includes a power source 1312. The power source 1312 may include, but is not restricted to, a battery for supplying power to other components such as, but not limited to, the processor 1308 and the display 1310. The battery may be rechargeable or disposable. The power source 1312 may further include, but is not restricted to, a lithium ion battery, or a lithium ion polymer battery.

Further, in some embodiments, the portable electronic device 1300 may include a test strip container 1302 for storing multiple test strips. The test strip container 1302 may be of variable dimensions to store multiple glucose test strips of variable sizes and shapes provided by different manufacturers. In some embodiments, the test strip container 1302 may be detachably attached to the portable electronic device 1300.

The portable electronic device 1300 further includes a lancet container 1304 for storing multiple lancets. The lancet container 1304 can be of variable dimensions to store multiple lancets of variable sizes and shapes. In some embodiments, the lancet container 1304 can be detachably attached to the portable electronic device 1300.

In some embodiments, the portable electronic device 1300 can be detachably associated with an electronic device including, but not limited to, a cell phone, or a tablet.

In some embodiments, the portable electronic device 1300 may be a wearable device. The portable electronic device 1300 includes a band 1322 for detachably coupling the housing to a user.

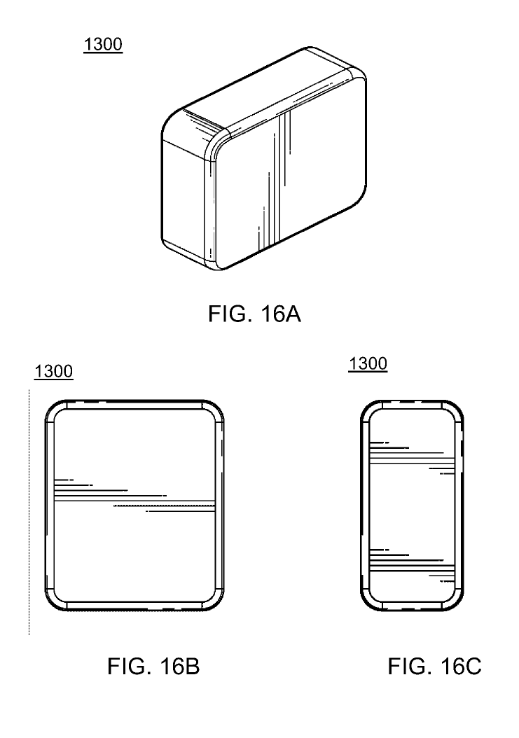

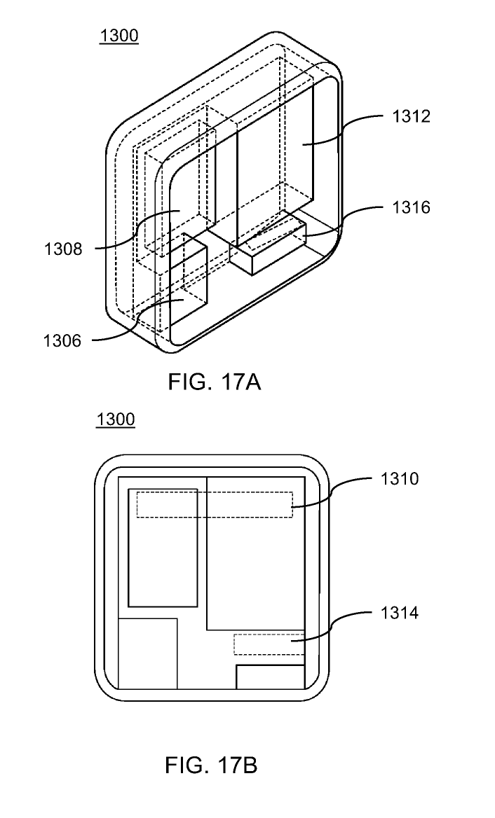

FIGS. 16A, 16B and 16C illustrate isometric, front and side views of the portable electronic device 1300, respectively. FIGS. 17A and 17B illustrate cross-sectional views of the portable electronic device 1300 for analyzing an analyte. The portable electronic device 1300 includes a housing having the interface port 1306 disposed at a first end of the housing for positioning a blood glucose measuring test strip within the housing. Further, the portable electronic device 1300 includes the lancet port 1316 disposed at a second end of the housing for positioning a lancet within the housing.

The portable electronic device 1300 also includes a processor 1308 for calculating and transmitting data obtained by analyzing an analyte. The portable electronic device 1300 includes a display 1310 disposed at the housing for displaying the data, a power source 1312, and a charging port 1314 for charging the power source 1312.

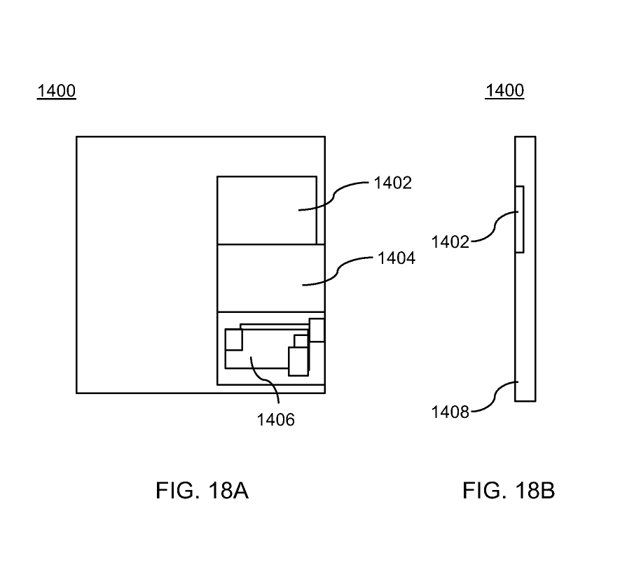

FIG. 18A illustrates a hardware block diagram of a portable electronic device 1400 for analyzing an analyte, according to an embodiment of the present invention. The portable electronic device 1400 includes a housing with a lancet holder 1402, a test strip holder 1404 and a circuit board 1406.

FIG. 18B illustrates another view of the portable electronic device 1400. The portable electronic device 1400 includes a test strip holder 1404 and a strip insertion hole or opening 1408. The lancet holder 1402 stores one or more lancets. The test strip holder stores one or more test strips. A user may prick his/her skin to extract drops blood using a lancet. The extracted drops of blood are placed on a test strip. The test strip is inserted in the strip insertion hole 1408.

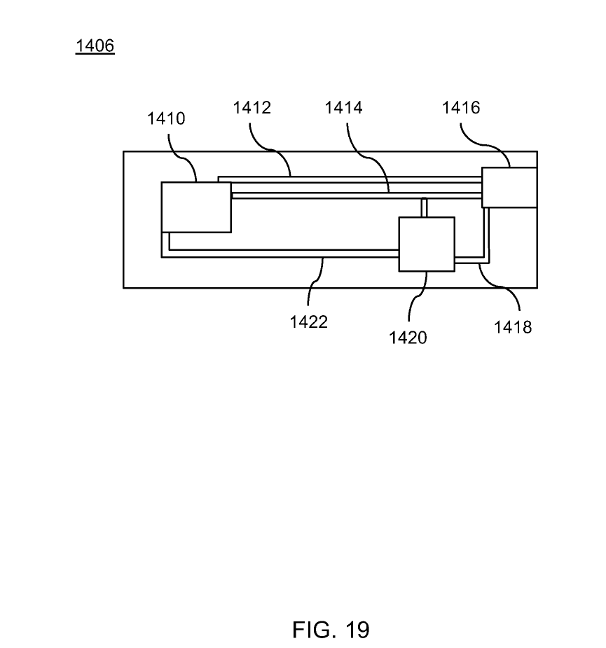

FIG. 19 illustrates a block diagram of the circuit board 1406 of the portable electronic device 1400. The circuit board 1406 includes a microprocessor 1410 for calculating and transmitting data obtained by analyzing an analyte. The circuit board further includes a strip interface 1416 and a battery holder 1420. The battery holder 1420 has a battery that supplies Direct Current (DC) for the microprocessor 1410 via a signal trace 1412 between the microprocessor 1410 and the strip interface 1416. Further, the direct current may be transferred via a DC common trace 1414 between the microprocessor 1410 and the strip interface 1416. Moreover, the signal trace 1412 can be carried out by segregating a first DC trace 1418 from the strip interface 1416 to the battery holder 1420 and a second DC trace 1422 from the battery in the battery holder 1420 to the circuit board 1408.

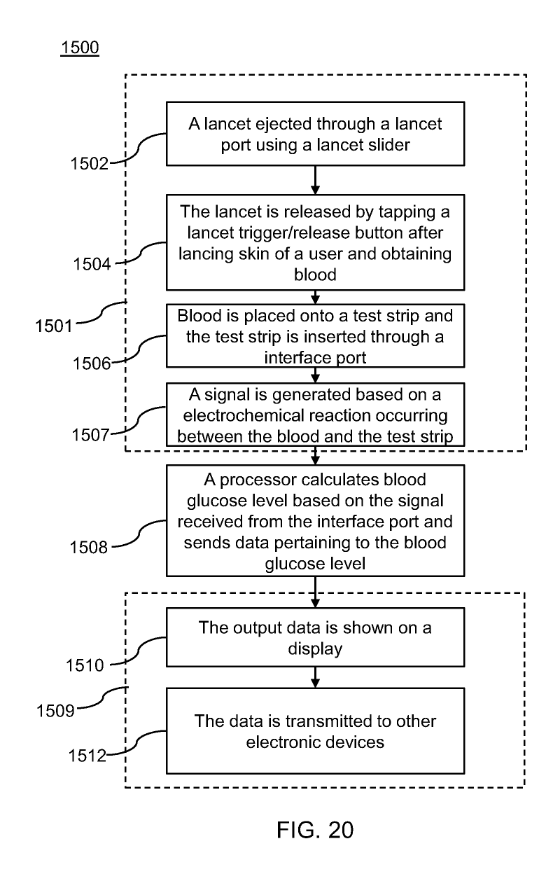

FIG. 20 illustrates a functional block diagram 1500 of the portable electronic device 1300, shown in FIG. 15. The block diagram 1800 illustrates functioning of major components of the portable electronic device 100 with their inter-linkage. The block diagram 1800 includes an input 1501, a processor 1508, such as the processor 1308 (shown in FIG. 15), and an output 1509.

The input 1501 includes a step 1502 for ejecting a lancet through the lancet port 1316 by using the lancet slider 1318. The lancet slider 1318 is used to lance skin of a user. At step 1504, the lancet is released by tapping the lancet trigger/release button 1320, to obtain blood. At step 1506, the blood is then placed onto a test strip that is further placed in the interface port 1306. At step 1507, a signal is generated based on an electrochemical reaction between the blood and the test strip. The signal is transmitted to the processor 1508 through the interface port 1306. The signal may be an electrical signal or a magnetic signal.

The signal from the input 1501 is further fed into the processor 1508 that calculates blood glucose level based on the signal and generates output data, i.e., the blood glucose level.

The output data is further transmitted to the output 1509, where the output data is displayed on the display 1310 (step 1510). In some embodiments of the present invention, the output data is transmitted to other electronic devices via Bluetooth™ (step 1512).

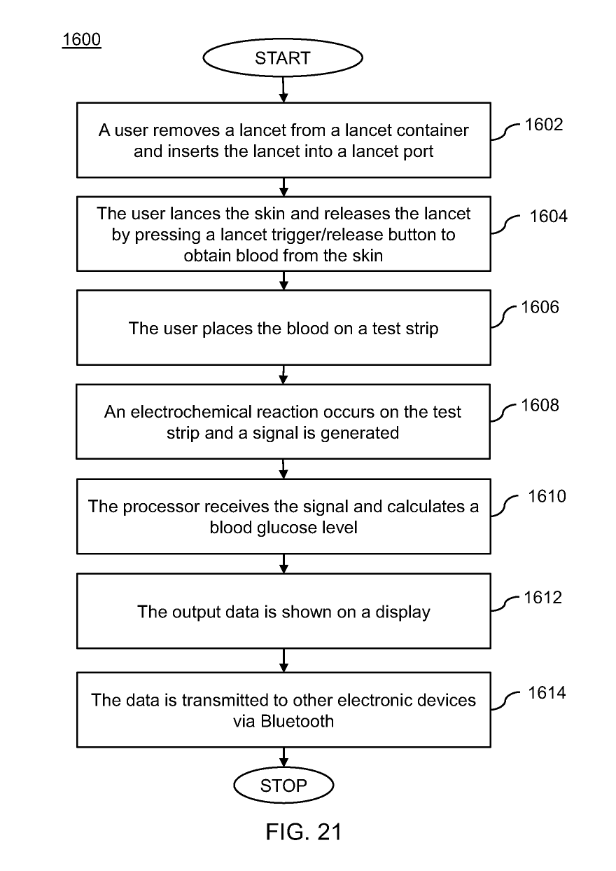

FIG. 21 illustrates a flowchart schematically outlining a method 1600 for analyzing an analyte by using the portable electronic device 1300, according to an embodiment of the present invention.

At step 1602, a user removes a lancet from a lancet container and inserts the lancet into the lancet port 1316 of the portable electronic device 1300.

At step 1604, the user lances the skin to obtain blood sample and then releases the lancet by pressing a lancet trigger/release button after obtaining blood. At step 1606, the user places the blood on a test strip.

At step 1608, the blood sample is placed on the glucose test strip and an electrochemical reaction occurs on the test strip. Based on the reaction, a signal is generated and transmitted to the processor 1308.

Thereafter, at step 1610, the processor 1308 receives the signal that is further used to calculate a blood glucose level. The calculated blood glucose level is transmitted to an output unit.

Next, at step 1612, the calculated blood glucose level is shown on a display of the portable electronic device 1300. Further, at step 1314, the user's calculated blood glucose level can also be transmitted to other electronic devices (e.g., a smartphone) via short wave communication signals such as, but not restricted to, Bluetooth™.

In an exemplary scenario, the working of the portable electronic device is explained. A user removes a test strip and a lancet from either the respective containers or the lancet device and inserts the lancet into a lancet port and the glucose test strip into the glucose test strip interface port. The user slides back the lancet slider on the glucose monitoring device face. There are multiple settings associated with the lancet slider. The multiple settings allow varying tensions applied to the lancet. For example, the more the user slides the slider, more tension is applied to the lancet, and harder the lancet pierces the skin. The user then taps the lancet release/trigger button to discharge the lancet. The user then places a small amount of blood onto the test strip. A voltage is generated by a chemical reaction caused by an interface of glucose levels in the user's blood, and a chemically treated metal on the glucose test strip. The processor then reads the voltage and calculates the user's blood glucose level that is displayed onto a display of the glucose monitoring device. This information is also transmitted to the user's cell phone, tablet or computer.

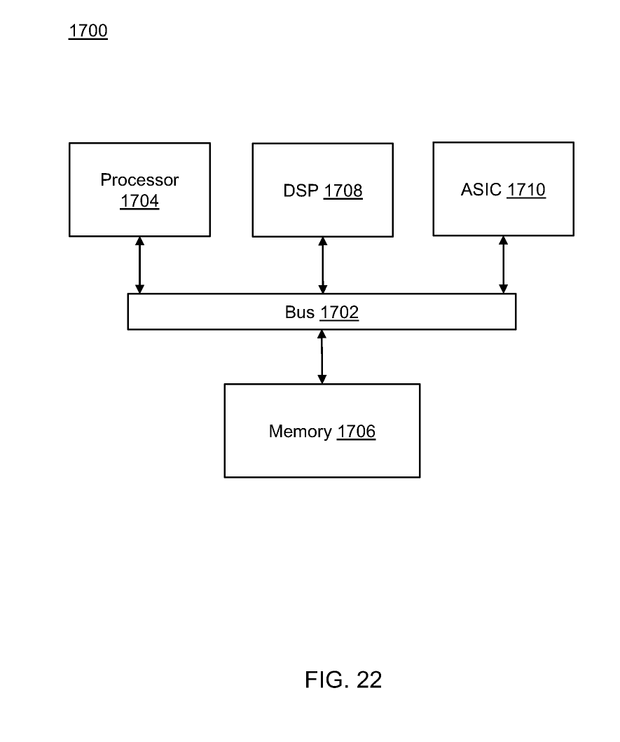

FIG. 22 illustrates a chipset 1700 upon which an embodiment of the invention may be implemented. The chipset 1700 is programmed to process and transmit glucose level data in a bandwidth efficient manner as described herein and includes, for instance, the processor and memory components incorporated in one or more physical packages. By way of example, a physical package of the chip set 1700 includes an arrangement of one or more materials, components, and/or wires on a structural assembly (e.g., a baseboard) to provide characteristics, such as physical strength, conservation of size, and/or limitation of electrical interaction. It is contemplated that in certain embodiments the chipset 1700 can be implemented in a single chip. The chip set 1700, or a portion thereof, constitutes a means for determining blood glucose level of a user.

In one embodiment, the chipset 1700 includes a communication mechanism, such as a bus 1702, for passing the data among the components of the chip set 1700. A processor 1704 is coupled to the bus 1702. The processor 1704 executes instructions and processes the data stored in a memory 1706. The processor 1704 may include one or more processing cores with each core configured to perform independently. A multi-core processor enables multiprocessing within a single physical package. Examples of a multi-core processor include two, four, eight, or greater numbers of processing cores. Alternatively, the processor 1704 may include microprocessors configured in tandem via the bus 1702 to enable independent execution of instructions, pipelining, and multithreading. The processor 1704 may also be accompanied with specialized components to perform certain processing functions and tasks such as a Digital Signal Processor (DSP) 1708, or an Application-Specific Integrated Circuit (ASIC) 1710. The DSP 1708 processes real-world signals independently of the processor 1704. Similarly, the ASIC 1710 can be configured to perform specialized functions not easily performed by a more general purpose processor. Other specialized components to aid in performing the inventive functions described herein may include, but not restricted to, Field Programmable Gate Arrays (FPGA), controllers, or other special-purpose computer chips.

The processor 1704 and accompanying components are connected to the memory 1706 via the bus 1702. The memory 1706 includes both dynamic memory (e.g., Random Access Memory (RAM), magnetic disk, writable optical disk, etc.) and static memory (e.g., Read Only Memory (ROM), a compact disc (CD) etc.) for storing executable instructions that when executed perform the inventive steps described herein to process and transmit sensor data in a bandwidth efficient manner. The memory 1706 also stores the data associated with or generated by the execution of the inventive steps.

The present invention, in various embodiments, configurations, and aspects, includes components, methods, processes, systems and/or apparatus substantially as depicted and described herein, including various embodiments, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and/or reducing cost of implementation.

The foregoing discussion of the present invention has been presented for purposes of illustration and description. It is not intended to limit the present invention to the form or forms disclosed herein. In the foregoing Detailed Description, for example, various features of the present invention are grouped together in one or more embodiments, configurations, or aspects for the purpose of streamlining the disclosure. The features of the embodiments, configurations, or aspects may be combined in alternate embodiments, configurations, or aspects other than those discussed above. This method of disclosure is not to be interpreted as reflecting an intention the present invention requires more features than are expressly recited in each claim.

Moreover, though the description of the present invention has included description of one or more embodiments, configurations, or aspects and certain variations and modifications, other variations, combinations, and modifications are within the scope of the present invention, e.g., as may be within the skill and knowledge of those in the art, after understanding the present disclosure. It is intended to obtain rights which include alternative embodiments, configurations, or aspects to the extent permitted, including alternate, interchangeable and/ or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A portable electronic device to analyze an analyte, the portable electronic device comprising:
a housing;
an adapter detachably coupled to the housing, the adapter comprising:
a body defining an opening to receive a test strip; and
an interface port disposed within the body, wherein the interface port is configured to read a signal from the test strip; and
a processor disposed in the housing and communicably coupled to the interface port, the processor configured to determine a parameter of the analyte based on the signal received from the interface port.

2. The portable electronic device of claim 1, wherein the adapter is selected from a plurality of adapters, each of the plurality of adapters having different physical dimensions of the interface port.

3. The portable electronic device of claim 2, wherein the housing further includes an adapter port coupleable with each of the plurality of adapters, the adapter port being communicably coupled to the processor.

4. The portable electronic device of claim 2, wherein each of the plurality of adapters is configured to interface with a corresponding type of test strip.

5. The portable electronic device of claim 1, wherein the housing further comprises:
a test strip compartment to store a test strip;
a lancet compartment to store a lancet; and
a needle compartment to store a lancet needle.

6. The portable electronic device of claim 5, further comprising a storage cover detachably coupled to the housing to cover the test strip compartment, the lancet compartment and the needle compartment.

7. The portable electronic device of claim 1, wherein the housing further comprises a case body detachably coupled to a mobile device.

8. The portable electronic device of claim 1, wherein the housing further comprises a clip to detachably couple the housing to a case of a mobile device.

9. The portable electronic device of claim 1, wherein the housing further comprises a band to detachably couple the housing to a user.

10. The portable electronic device of claim 1, wherein the processor is further configured to:
generate a user interface on a display; and
display indicia indicative of the parameter of the analyte on the user interface.

11. A system to analyze an analyte, the system comprising:
a portable electronic device comprising:
a housing;
an adapter detachably coupled to the housing, the adapter comprising:
a body defining an opening to receive a test strip; and
an interface port disposed within the body, wherein the interface port is configured to read a signal from the test strip; and
a processor disposed in the housing and communicably coupled to the interface port, the processor configured to determine a parameter of the analyte based on the signal received from the interface port; and
a mobile device communicably coupled to the portable electronic device, the mobile device configured to display indicia indicative of the parameter of the analyte on a user interface.

12. The system of claim 11, wherein the adapter is selected from a plurality of adapters, each of the plurality of adapters having different physical dimensions of the interface port.

13. The system of claim 12, wherein the housing further includes an adapter port coupleable with each of the plurality of adapters, the adapter port being communicably coupled to the processor.

14. The system of claim 12, wherein each of the plurality of adapters is configured to interface with a corresponding type of test strip.

15. The system of claim 11, wherein the housing further comprises a case body configured to be detachably coupled to the mobile device.

16. The system of claim 11, wherein the housing further comprises a clip to detachably couple the housing to a case of the portable electronic device.

17. The system of claim 11, wherein the housing further comprises a band to detachably couple the housing to a user.

18. A system to analyze an analyte, the system comprising:
a portable electronic device comprising:
a housing comprising an adapter port; and
a processor disposed in the housing and communicably coupled to the adapter port, the processor configured to determine a parameter of the analyte based on a signal received from the adapter port; and
a plurality of adapters, each of the plurality of adapters coupleable to the adapter port of the housing, each of the plurality of adapters comprising:
a body defining an opening to receive a test strip;
an interface port disposed within the body, wherein the interface port is configured to read the signal from the test strip; and
an electronic circuit configured to transmit the signal to the adapter port,
wherein each of the plurality of adapters has different physical dimensions of the interface port.

19. The system of claim 18, wherein each of the plurality of adapters is configured to interface with a corresponding type of test strip.

20. The system of claim 18, wherein the housing further comprises a case body configured to be detachably coupled to a mobile device.
